(19) United States
(12) Patent Application Publication
(10) Pub. No.: US 2020/0163575 A1
(43) Pub. Date: May 28, 2020

(54) ANALYSING PHONOCARDIOGRAM AND ELECTROCARDIOGRAM DATA FROM A PORTABLE SENSOR DEVICE

(71) Applicant: Coala-Life AB, Stockholm (SE)

(72) Inventors: Magnus Samuelsson, Stockholm (SE); Phillip Siberg, Stockholm (SE); Martin Stridh, Hjärup (SE); Jacob Söndergaard Svensson, Klagshamn (SE)

(73) Assignee: Coala-Life AB, Stockholm (SE)

(21) Appl. No.: 16/620,640

(22) PCT Filed: Jun. 28, 2018
(86) PCT No.: PCT/SE2018/050708

§ 371 Date: Dec. 9, 2019

(30) Foreign Application Priority Data
Jun. 30, 2017 (SE) 1750858-1

(51) Int. Cl. A61B 5/0452; A61B 5/0404; A61B 7/04; G16H 40/67; G16H 50/30

(52) U.S. Cl. CPC A61B 5/04525; A61B 5/0404; A61B 5/0006; G16H 40/67; G16H 50/30; A61B 7/04

(57) ABSTRACT

It is presented a method for analysing heart data of a user. The method comprises the steps of: receiving phonocardiogram data from a portable sensor device; receiving electrocardiogram data from the portable sensor device, wherein the electrocardiogram data corresponds to the phonocardiogram data in time; dividing the phonocardiogram data in time segments based on cardiac cycles identified using at least one of the phonocardiogram data and the electrocardiogram data; dividing the electrocardiogram data in time segments corresponding to the time segments of the phonocardiogram data; and determining whether the heart is considered to need further examination or not based on only time segments of the phonocardiogram data and the electrocardiogram data where the quality of the phonocardiogram data is greater than a threshold level and the quality of the electrocardiogram data is greater than a threshold level.

### TECHNICAL FIELD

The invention relates to a method, an analysis device, a computer program and a computer program product analysing phonocardiogram and electrocardiogram data from a portable sensor device.

### BACKGROUND

ECG is an established technology where electric signals generated by the body of a patient are measured and analysed. Traditionally, a number of electrodes are placed on the body at various places. A conductive gel is used to provide better conductive contact between the electrode and the skin. The patient typically lies down for minutes when the ECG is taken. The data detected using the electrodes is recorded and can be analysed by a professional, such as a physician or trained nurse. Once the measurement procedure is done, the conductive gel is wiped off.

While having proved useful, the traditional way of obtaining an ECG is not optimal in all cases. For instance, such an ECG needs to be measured in a clinic and the procedure is messy for the patient.

Lately, portable sensor devices with integral electrodes for obtaining ECG data have been developed. These portable sensor devices allow users to capture ECG data at will and also without the use of conductive gel. This gives the user greater control over when to capture ECG data and also in a much more convenient and less messy way.

Such portable sensor devices can also be configured to measure phonocardiogram (PCG) data, i.e. sound data of the heart. However, PCG data captured by a portable device is susceptible to a noisier environment than e.g. in a clinic. Additionally, more noise can occur when an inexperienced user is using the portable sensor device to capture the PCG data, compared to when an experienced medical professional captures the PCG data.

The analysis of electrocardiogram data and the phonocardiogram data is complicated and any incorrect analysis should be avoided to the greatest extent possible, as this can affect the health of the user.

### SUMMARY

It is an object to improve the analysis of the combination of electrocardiogram data and phonocardiogram data.

According to a first aspect, it is presented a method for analysing heart data of a user. The method is performed in an analysis device and comprises the steps of: obtaining phonocardiogram data, representing audio data of activities of the heart, from a portable sensor device; obtaining electrocardiogram data, based on electrical signals measured by electrodes placed on the body of the user, from the portable sensor device, wherein the electrocardiogram data corresponds to the phonocardiogram data in time; dividing the phonocardiogram data in time segments based on cardiac cycles identified using at least one of the phonocardiogram data and the electrocardiogram data; dividing the electrocardiogram data in time segments corresponding to the time segments of the phonocardiogram data; and determining whether the heart is considered to need further examination or not based on only time segments of the phonocardiogram data and the electrocardiogram data where the quality of the phonocardiogram data is greater than a threshold level and the quality of the electrocardiogram data is greater than a threshold level.

The step of dividing may comprise dividing the phonocardiogram data in time segments based on cardiac cycles identified using the electrocardiogram data.

Each cardiac cycle may be made up of a plurality of time segments.

The step of determining whether the heart is considered to need further examination or not may comprise the step of: calculating a composite of data in for corresponding time segments in the cardiac cycles.

The step of determining whether the heart is considered to need further examination or not may comprise: determining a time between a peak in the electrocardiogram data and a peak in the phonocardiogram data.

The method may further comprise the step of: adjusting a gain applied for the phonocardiogram data based on the electrocardiogram data.

The step of determining whether the heart is considered to need further examination or not may comprise the step of: deriving a plurality of frequency components of the phonocardiogram data.

The step of determining whether the heart is considered to need further examination or not may comprise the steps of: determining whether there is a signal greater than a threshold level in a particular frequency component of the phonocardiogram data; determining that the heart is considered to need further examination when there is no signal greater than the threshold level in the particular frequency component; and analysing signal levels in other frequency components when there a signal greater than the threshold level in the particular frequency component.

The method may further comprise the step of: transmitting a signal to a device of the user containing information of whether the heart is considered to need further examination or not.

According to a second aspect, it is presented an analysis device for analysing heart data of a user. The analysis device comprises: a processor; and a memory storing instructions that, when executed by the processor, cause the analysis device to: obtain phonocardiogram data, representing audio data of activities of the heart, from a portable sensor device; obtain electrocardiogram data, based on electrical signals measured by electrodes placed on the body of the user, from the portable sensor device, wherein the electrocardiogram data corresponds to the phonocardiogram data in time; divide the phonocardiogram data in time segments based on cardiac cycles identified using at least one of the phonocardiogram data and the electrocardiogram data; divide the electrocardiogram data in time segments corresponding to the time segments of the phonocardiogram data; and determine whether the heart is considered to need further examination or not based on only time segments of the phonocardiogram data and the electrocardiogram data where the quality of the phonocardiogram data is greater than a threshold level and the quality of the electrocardiogram data is to greater than a threshold level.

According to a third aspect, it is presented a computer program for analysing heart data of a user. The computer program comprises computer program code which, when run on an analysis device causes the analysis device to: obtain phonocardiogram data, representing audio data of activities of the heart, from a portable sensor device; obtain electrocardiogram data, based on electrical signals measured by electrodes placed on the body of the user, from the portable sensor device, wherein the electrocardiogram data corresponds to the phonocardiogram data in time; divide the phonocardiogram data in time segments based on cardiac cycles identified using at least one of the phonocardiogram data and the electrocardiogram data; divide the electrocardiogram data in time segments corresponding to the time segments of the phonocardiogram data; and determine whether the heart is considered to need further examination or not based on only time segments of the phonocardiogram data and the electrocardiogram data where the quality of the phonocardiogram data is greater than a threshold level and the quality of the electrocardiogram data is greater than a threshold level.

According to a fourth aspect, it is presented a computer program product comprising a computer program according to the third aspect and a computer readable means on which the computer program is stored.

Generally, all terms used in the claims are to be interpreted according to their ordinary meaning in the technical field, unless explicitly defined otherwise herein. All references to “a/an/the element, apparatus, component, means, step, etc.” are to be interpreted openly as referring to at least one instance of the element, apparatus, component, means, step, etc., unless explicitly stated otherwise. The steps of any method disclosed herein do not have to be performed in the exact order disclosed, unless explicitly stated.

### BRIEF DESCRIPTION OF THE DRAWINGS

The invention is now described, by way of example, with reference to the accompanying drawings, in which:

FIGS. 1A-B are schematic diagrams illustrating an environment in which embodiments presented herein can be applied;

FIG. 2 is a schematic diagram illustrating when the portable sensor device is used to capture measurements for ECG;

FIGS. 3A-3B are schematic diagrams of views illustrating a physical representation of the portable sensor device according to one embodiment;

FIGS. 4A-B are schematic graphs illustrating how phonocardiogram data and electrocardiogram data can be used according to some embodiments;

FIG. 5 is a schematic diagram illustrating the analysis device of FIGS. 1A-B according to one embodiment;

FIGS. 6A-B are flow charts illustrating embodiments of methods for analysing heart data of a user, the methods being performed in the analysis device of FIGS. 1A-B; and

FIG. 7 shows one example of a computer program product comprising computer readable means.

### DETAILED DESCRIPTION

The invention will now be described more fully hereinafter with reference to the accompanying drawings, in which certain embodiments of the invention are shown. This invention may, however, be embodied in many different forms and should not be construed as limited to the embodiments set forth herein; rather, these embodiments are provided by way of example so that this disclosure will be thorough and complete, and will fully convey the scope of the invention to those skilled in the art. Like numbers refer to like elements throughout the description.

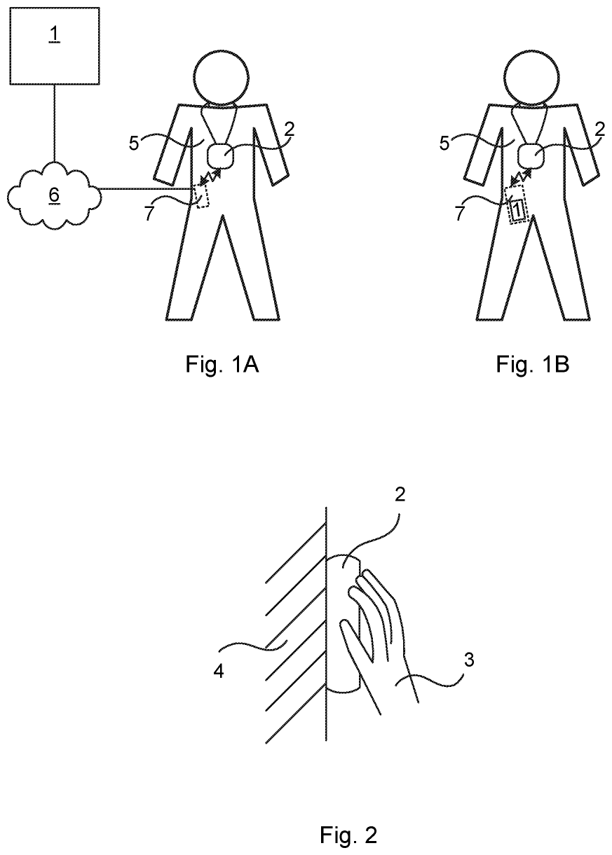

FIGS. 1A-B are schematic diagrams illustrating an environment in which embodiments presented herein can be applied.

Looking first to FIG. 1A, it is here shown a user 5 carrying a portable sensor device 2 in a necklace strap. The portable sensor device can be carried in any other way, e.g. in a pocket or in a handbag. The user 5 also carries a smartphone 7 e.g. in a pocket. The portable sensor device 2 and the smartphone 7 can communicate over any suitable wireless interface, e.g. using Bluetooth or Bluetooth Low Energy (BLE), ZigBee, any of the IEEE 802.11x standards (also known as WiFi), etc.

The smartphone 7 is also connected to a wide area network 6, such as the Internet, e.g. via WiFi or a cellular network, to allow communication with an analysis device 1, here in the form of a server. The portable sensor device 2 captures ECG data and PCG data and sends this data, via the smartphone 7, to the analysis device 1. This allows the analysis device 1 to determine whether the heart of the user 5 can be considered to be in a normal state or whether the heart needs further examination based on the PCG data and the ECG data captured by the portable sensor device 2. Further investigation can be determined to be needed e.g. if any abnormal heart condition cannot be ruled out. It is to be noted that even if further investigation is to be performed, the heart can in fact be normal, i.e. non-pathological.

In FIG. 1B, the smartphone 7 contains the analysis device 1. In this way, the analysis can be performed locally, without the need for immediate access to the wide area network.

Alternatively, the analysis device can form part of the portable sensor device 2 (not shown). In such a case, the portable sensor 2 can also perform the functions of the smartphone 7.

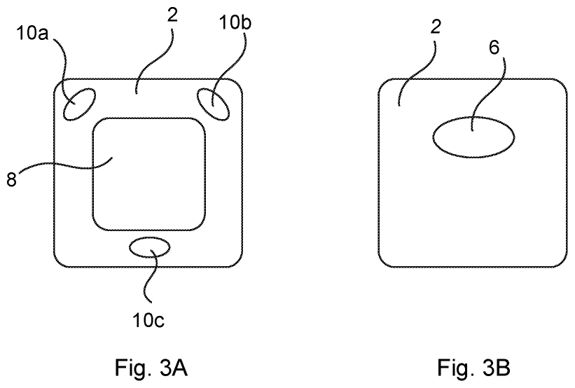

FIG. 2 is a schematic diagram illustrating when the portable sensor device 2 of FIG. 1 is used to capture measurements for ECG and for PCG. In order to capture measurements for ECG and for PCG, the portable sensor device 2 is placed on the skin of the body 2 of the user, close to the heart of the user. The user holds the portable sensor device 2 in place using a hand 3. It is to be noted that there are no loose electrodes for the ECG measurement. Instead, the electrodes (as shown in FIG. 3A and described below) are provided integral to the portable sensor device 2. Hence, the measurement for the ECG is captured simply by the user holding the portable sensor device 2 in contact with the skin of the body 2. Moreover, the PCG measurements can be performed concurrently with the ECG measurements. In this way, the ECG and the PCG for the same time can be analysed to improve analysis capabilities of the state of the heart of the user.

FIGS. 3A-3B are schematic diagrams of views illustrating a physical representation of the portable sensor device 2 of FIG. 1 according to one embodiment.

In FIG. 3A, a bottom view of the portable sensor device 2 is shown. There are a first electrode 10a, a second electrode 10b and a third electrode 10c. In order to capture the ECG data, the electrodes 10a-c are placed on the casing of the portable sensor device 2 such that when the user places the portable sensor device 2 on the skin, all electrodes 10a-c are in contact with the skin. It is to be noted that the portable sensor device 2 could also be provided with two electrodes, four electrodes or any other suitable number of electrodes. Using the electrodes, one or more analogue ECG signals are captured. The analogue ECG signals are converted to digital ECG signals using an analogue to digital (A/D) converter. The digital ECG signal is then sent to the analysis device for analysis together with the PCG signal.

Additionally, a transducer 8, e.g. in the form of a microphone, is provided to convert sound captured by the body into electric analogue PCG signals. The analogue PCG signals are converted to digital PCG signals using an A/D converter. The digital PCG signal is then sent to the analysis device for analysis together with the ECG signal

In FIG. 3B, a top view of the portable sensor device 2 is shown. Here, a user interface element 4 in form of a push button is shown. The push button can e.g. be used by the user to indicate when to start a measurement of ECG data and PCG data. It is to be noted that other user interface elements can be provided (not shown), e.g. more push buttons, Light Emitting Diodes (LEDs), a display, a speaker, a user microphone, etc.

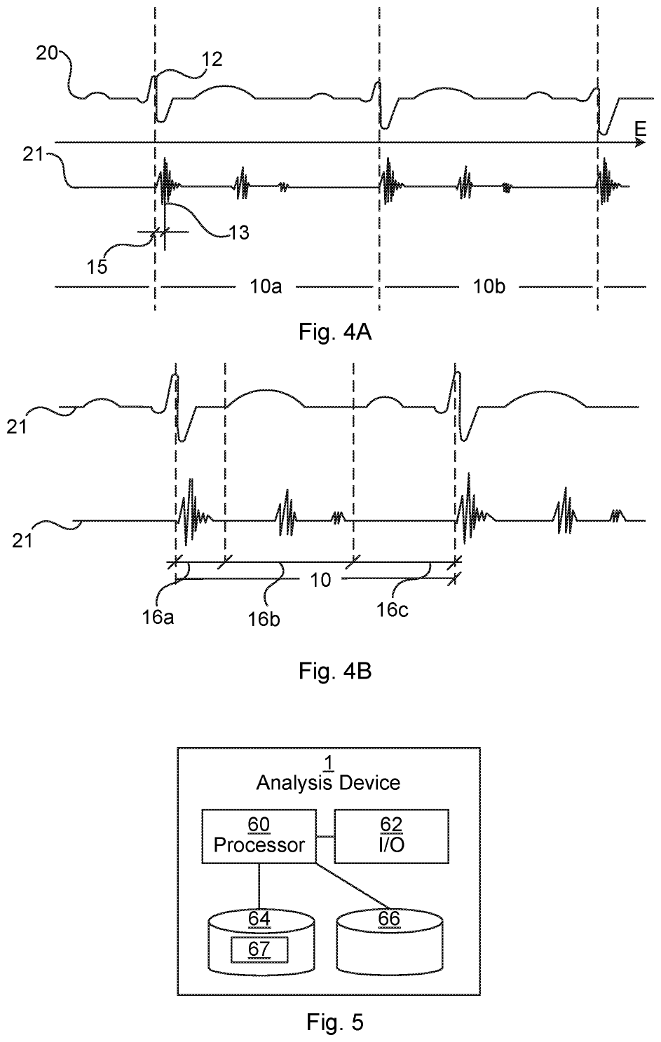

FIGS. 4A-B are schematic graphs illustrating how phonocardiogram data and electrocardiogram data can be used according to some embodiments. First, the graph of FIG. 4A will be described. Both an ECG signal 20 and a PCG signal 21 are shown, along a common timeline from left to right. There are here two full cardiac cycles 10a, 10b. It is to be noted that the start and end of each cardiac cycle 10a, 10b is not important, as long as the start and the end of each cardiac cycle 10a, 10b are at equivalent points in the cardiac cycles.

One measurement which can be used in the analysis of the ECG data and the PCG data is a time measurement 15 between a peak 12 in the ECG data and a peak 13 in the PCG data. The peak 12 in the ECG data is the peak in the QRS complex, representing the rapid depolarization of the right and left ventricles. The peak 13 in the PCG data is the sound of when valves are closed, which is the peak of the PCG data with the greatest amplitude.

The time measurement 15 can be expressed as a percentage of the average cardiac cycle. When this measurement 15 is excessive, this indicates an abnormal condition which should be investigated further.

Looking now to FIG. 4B, it is here shown how a cardiac cycle 10 is divided into three segments 16a, 16b and 16c. All cardiac cycles are divided in the same way. Consequently, corresponding segments of consecutive cardiac cycles can be used for composite analysis (e.g. average, median, weighted average, etc.) to improve signal quality. The segmenting of each cardiac cycle can be based on events in the ECG signal 20 or the PCG signal 21.

It is to be noted that there can be any number of segments in a cardiac cycle and the segments can be provided at different sections than what is shown in FIG. 4B, as long as the division into segments is consistent across cardiac cycles.

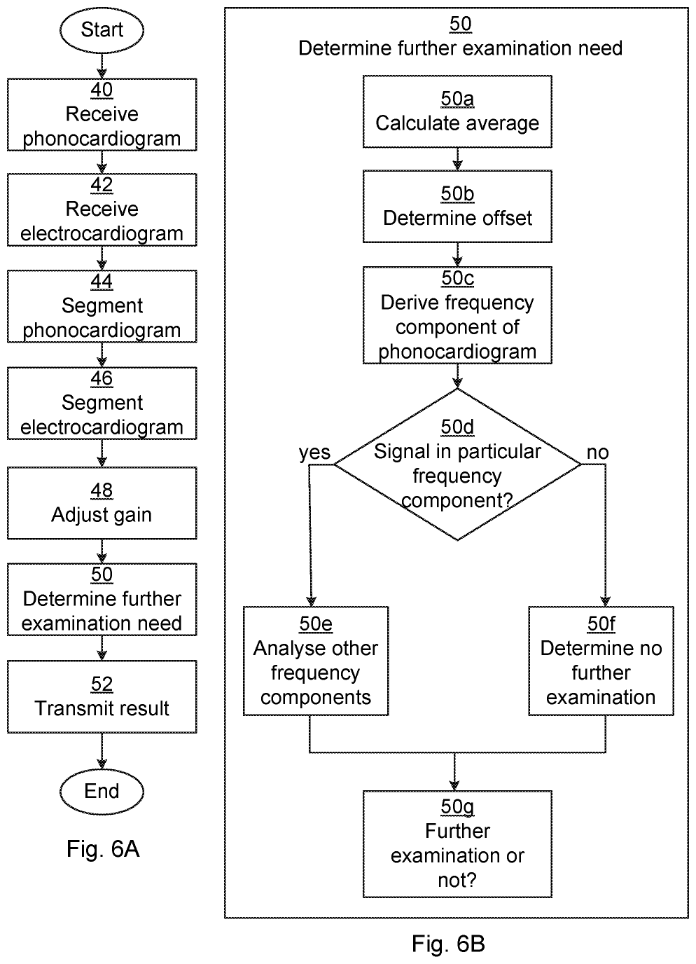

FIG. 5 is a schematic diagram illustrating the analysis device 1 of FIG. 1 according to one embodiment. As shown in FIGS. 1A-B, the analysis device can be implemented as part of a server or as part of a user device, such as a smartphone or alternatively as part of the portable sensor device. A processor 60 is provided using any combination of one or more of a suitable central processing unit (CPU), multiprocessor, microcontroller, digital signal processor (DSP), application specific integrated circuit etc., capable of executing software instructions 67 stored in a memory 64, which can thus be a computer program product. The processor 60 can be configured to execute the method described with reference to FIGS. 6A-B below.

The memory 64 can be any combination of read and write memory (RAM) and read only memory (ROM). The memory 64 also comprises persistent storage, which, for example, can be any single one or combination of magnetic memory, optical memory, solid state memory or even remotely mounted memory.

A data memory 66 is also provided for reading and/or storing data during execution of software instructions in the processor 60. The data memory 66 can be any combination of read and write memory (RAM) and read only memory (ROM).

The analysis device 1 further comprises an I/O interface 62 for communicating with other external entities, such as the smartphone 7 of the user using Internet Protocol (IP) over the wide area network 6.

Other components of the analysis device are omitted in order not to obscure the concepts presented herein

FIGS. 6A-B are flow charts illustrating embodiments of methods for analysing heart data of a user, the methods being performed in the analysis device of FIG. 1.

In an obtain phonocardiogram data step 40, PCG data is obtained from a portable sensor device. As explained above, the PCG data represents audio data of activities of the heart. The PCG data can be the digital PCG signals described above. The phonocardiogram data can be received from the portable measurement device.

In an obtain electrocardiogram data step 42, ECG data is obtained from the portable sensor device. As explained above, the ECG data is based on electrical signals measured by electrodes placed on the body of the user. The ECG data corresponds to the PCG data in time. The ECG data can be the digital ECG data described above. The electrocardiogram data can be received from the portable measurement device.

In a segment phonocardiogram data step 44, the PCG data is divided in time segments based on cardiac cycles identified using at least one of the PCG data and the ECG data. Optionally, each cardiac cycle is made up of a plurality of time segments, as shown in FIG. 4B and explained above. The time segments may be based on events in cardiac cycles identified using the electrocardiogram data, since cardiac events are often more robustly identifiable using the electrocardiogram data. Such events can e.g. be the P wave, QRS complex, T wave, U wave, etc., events which are known in the art per se and are easily identifiable

In a segment electrocardiogram data step 46, the ECG data is divided in time segments corresponding to the time segments of the PCG data. In other words, within a single cardiac cycle there are corresponding time segments in the ECG data and the PCG data.

In an optional adjust gain step 48, a gain applied for the PCG data is adjusted based on the ECG data. This allows the gain for the PCG data to be increased in sections when the PCG signal is expected to be low to capture details in the PCG signal. Also, the gain of the PCG data is then decreased in sections when the PCG signal is expected to be high to be able to capture the entire dynamic range of the signal. In other words, using the ECG data for the gain of the PCG data, both dynamic range and low level detail.

In a determine further examination need step 50, the analysis device determines whether the heart is considered to need further examination or not, based on only time segments of the PCG data and the ECG data where the quality of the PCG data is greater than a threshold level and the quality of the ECG data is greater than a threshold level. In other words, time segments where there is excessive interference or noise are discarded in the analysis. Particularly when combined with the optional composite calculation described below, the discarding of low quality segments increases overall signal quality, which can be applied for both PCG data and ECG data. Since interference can be short in duration, by discarding only time segments where there is low quality, other segments of the same cardiac cycle can be used and contribute to the analysis. The quality can e.g. be measured as a signal to noise ratio (SNR) or signal to noise and interference ratio (SINR), and the threshold level can be a specific numerical value of SNR or SINR. In one embodiment, the quality of ECG is quantified using a quality index. The quality index is based on identification of heart events, such as contractions, from the ECG data. Based on the identification, an ideal ECG signal is synthesised. The ECG data is then compared with the ideal ECG signal and its deviation is quantified, e.g. using RMS (Root Mean Square). The quantified deviation can thus function as a quality index. The quality of the PCH data can be quantified in an according way. In one embodiment, the quality is determined based on a set of quality criteria. Such quality criteria can include similarity between beats, likelihood of missed/extra detections, average rate and rhythm variability.

In an optional transmit result step 52, a signal is transmitted to a device of the user containing information of whether the heart is considered to need further examination or not. For instance, the signal can be transmitted to the smartphone of the user using IP over the wide area network. This allows the smartphone to display the result of the analysis to the user, indicating to the user whether the heart is considered to need further examination or whether the user should be investigated further to determine the user's heart condition.

Since the phonocardiogram data is more susceptible to noise than the electrocardiogram data, a better analysis is achieved by correlating the two types of data. This is particularly true when the data is captured using a portable sensor device, which might be used in a noisy environment. Moreover, the end user handling the portable sensor device may not be a trained medical professional, which may result in even more noise in the phonocardiogram data.

Looking now to FIG. 6B, this shows optional steps forming part of the determine further examination need step 50 of FIG. 6A.

In an optional calculate average step 50a, a composite of data in corresponding time segments in the cardiac cycles is calculated. The composite can e.g. be calculated by averaging, by obtaining a median value or calculating a weighted average (where e.g. extremes are omitted). When this is performed over many samples, noise or interference in individual cardiac cycles are reduced in intensity. Moreover, corresponding time segments can be of different duration in different cardiac cycles as long as the correspondence of the time segments in the cardiac cycle is ensured. In this way, signals for several cardiac cycles can form base for the analysis even when there is an irregular heart rhythm. Corresponding time segments can be determined by matching signals of similar patterns (see e.g. segments 16a-c of FIG. 4B, which is explained above) and/or of specific durations.

In an optional determine offset step 50b, a time between a peak in the ECG data and a peak in the PCG data is determined, as explained above for the time 15 with reference to FIG. 4A.

In an optional derive frequency components of phonocardiogram step 50c, a plurality of frequency components of the PCG data are derived. This can e.g. be done using fast Fourier transform (FFT) or wavelet analysis.

In an optional conditional signal in 1^{st} frequency component step 50d, the analysis device determines whether there is a signal greater than a threshold level in a particular frequency component of the PCG data. For instance, a heart murmur are sounds of relatively high frequency. Optionally, the duration of the signal in the particular frequency must also be longer than a specified duration.

If a signal greater than the threshold level in a particular frequency component is determined, the method proceeds to an optional determine no further examination step 50f. Otherwise, the method proceeds to an optional analyse other frequency components step 50e. Optionally, if the signal in the particular frequency component is constant throughout the heart cycle, this is typically not of physiological origin and is interpreted as background noise, and the method proceeds to the determine no further examination step 50f. Alternatively, the constant frequency component can indicate a low quality of the time segment and whereby the time segment could be disregarded.

In the optional analyse other frequency components step 50e, signal levels in other frequency components are analysed when there a signal greater than the threshold level in the particular frequency component.

In the optional determine no further examination step 50f, the analysis device determines that the heart is considered to need further examination when there is no signal greater than the threshold level in the particular frequency component.

In an optional further examination or not step 50g, the analysis device determines whether the heart is considered to need further examination or not based on the previous steps.

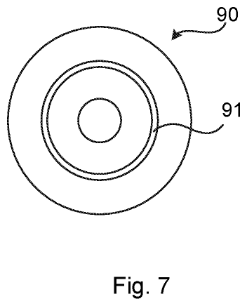

FIG. 7 shows one example of a computer program product comprising computer readable means. On this computer readable means a computer program 91 can be stored, which computer program can cause a processor to execute a method according to embodiments described herein. In this example, the computer program product is an optical disc, such as a CD (compact disc) or a DVD (digital versatile disc) or a Blu-Ray disc. As explained above, the computer program product could also be embodied in a memory of a device, such as the computer program product 64 of FIG. 5. While the computer program 91 is here schematically shown as a track on the depicted optical disk, the computer program can be stored in any way which is suitable for the computer program product, such as a removable solid state memory, e.g. a Universal Serial Bus (USB) drive.

The invention has mainly been described above with reference to a few embodiments. However, as is readily appreciated by a person skilled in the art, other embodiments than the ones disclosed above are equally possible within the scope of the invention, as defined by the appended patent claims.

What is claimed is:

1. A method for analysing heart data of a user, the method being performed in an analysis device and comprising the steps of:
obtaining phonocardiogram data, representing audio data of activities of the heart, from a portable sensor device;
obtaining electrocardiogram data, based on electrical signals measured by electrodes placed on the body of the user, from the portable sensor device, wherein the electrocardiogram data corresponds to the phonocardiogram data in time;
dividing the phonocardiogram data in time segments based on cardiac cycles identified using at least one of the phonocardiogram data and the electrocardiogram data;
dividing the electrocardiogram data in time segments corresponding to the time segments of the phonocardiogram data; and
determining whether the heart is considered to need further examination or not based on only time segments of the phonocardiogram data and the electrocardiogram data where the quality of the phonocardiogram data is greater than a threshold level and the quality of the electrocardiogram data is greater than a threshold level.

2. The method according to claim 1, wherein the step of dividing the phonocardiogram data comprises dividing the phonocardiogram data in time segments based on cardiac cycles identified using the electrocardiogram data.

3. The method according to claim 1, wherein each cardiac cycle is made up of a plurality of time segments.

4. The method according to claim 1, wherein the step of determining whether the heart is considered to need further examination or not comprises the step of:
calculating a composite of data in for corresponding time segments in the cardiac cycles.

5. The method according to claim 1, wherein the step of determining whether the heart is considered to need further examination or not comprises:
determining a time between a peak in the electrocardiogram data and a peak in the phonocardiogram data.

6. The method according to claim 1, further comprising the step of:
adjusting a gain applied for the phonocardiogram data based on the electrocardiogram data.

7. The method according to claim 1, wherein the step of determining whether the heart is considered to need further examination or not comprises the step of:
deriving a plurality of frequency components of the phonocardiogram data.

8. The method according to any claim 7, wherein the step of determining whether the heart is considered to need further examination or not comprises the steps of:
determining whether there is a signal greater than a threshold level in a particular frequency component of the phonocardiogram data;
determining that the heart is considered to need further examination when there is no signal greater than the threshold level in the particular frequency component; and
analysing signal levels in other frequency components when there a signal greater than the threshold level in the particular frequency component.

9. The method according to claim 1, further comprising the step of:
transmitting a signal to a device of the user containing information of whether the heart is considered to need further examination or not.

10. An analysis device for analysing heart data of a user, the analysis device comprising:
a processor; and
a memory storing instructions that, when executed by the processor, cause the analysis device to:
obtain phonocardiogram data, representing audio data of activities of the heart, from a portable sensor device;
obtain electrocardiogram data, based on electrical signals measured by electrodes placed on the body of the user, from the portable sensor device, wherein the electrocardiogram data corresponds to the phonocardiogram data in time;
divide the phonocardiogram data in time segments based on cardiac cycles identified using at least one of the phonocardiogram data and the electrocardiogram data;
divide the electrocardiogram data in time segments corresponding to the time segments of the phonocardiogram data; and
determine whether the heart is considered to need further examination or not based on only time segments of the phonocardiogram data and the electrocardiogram data where the quality of the phonocardiogram data is greater than a threshold level and the quality of the electrocardiogram data is greater than a threshold level.

11. A non-transitory, computer-readable storage medium storing instructions that when executed by a computer cause the computer to perform a method for analysing heart data for a user, wherein the method comprises:
obtaining phonocardiogram data, representing audio data of activities of the heart, from a portable sensor device;
obtaining electrocardiogram data, based on electrical signals measured by electrodes placed on the body of the user, from the portable sensor device, wherein the electrocardiogram data corresponds to the phonocardiogram data in time;
dividing the phonocardiogram data in time segments based on cardiac cycles identified using at least one of the phonocardiogram data and the electrocardiogram data;
dividing the electrocardiogram data in time segments corresponding to the time segments of the phonocardiogram data; and
determining whether the heart is considered to need further examination or not based on only time segments of the phonocardiogram data and the electrocardiogram data where the quality of the phonocardiogram data is greater than a threshold level and the quality of the electrocardiogram data is greater than a threshold level.

12. The non-transitory, computer-readable storage medium of claim 11, wherein dividing the phonocardiogram data comprises dividing the phonocardiogram data in time segments based on cardiac cycles identified using the electrocardiogram data.

13. The non-transitory, computer-readable storage medium according to claim 11, wherein each cardiac cycle is made up of a plurality of time segments.

14. The non-transitory, computer-readable storage medium according to claim 11, wherein the determining whether the heart is considered to need further examination or not comprises:
calculating a composite of data in for corresponding time segments in the cardiac cycles.

15. The non-transitory, computer-readable storage medium according to claim 11, wherein determining whether the heart is considered to need further examination or not comprises:
determining a time between a peak in the electrocardiogram data and a peak in the phonocardiogram data.

16. The analysis device of claim 10, wherein while dividing the phonocardiogram data, the analysis device divides the phonocardiogram data in time segments based on cardiac cycles identified using the electrocardiogram data.

17. The analysis device of claim 10, wherein each cardiac cycle is made up of a plurality of time segments.

18. The analysis device of claim 10, wherein while determining whether the heart is considered to need further examination or not, the analysis device additionally calculates a composite of data in for corresponding time segments in the cardiac cycles.

19. The analysis device of claim 10, wherein while determining whether the heart is considered to need further examination or not, the analysis device determines a time between a peak in the electrocardiogram data and a peak in the phonocardiogram data.

20. The analysis device of claim 10, wherein the instructions additionally cause the analysis device to adjust a gain applied to the phonocardiogram data based on the electrocardiogram data.
